(19) United States
(12) Patent Application Publication
(10) Pub. No.: US 2026/0318902 A1
(43) Pub. Date: Oct. 15, 2026

(54) MICRO-ELECTRODE ARRAYS FOR ELECTRICAL INTERFACING

(71) Applicant: Neural Automations Ltd., Gatineau (CA)

(72) Inventor: Yi GUO, Gatineau (CA)

(21) Appl. No.: 19/120,080

(22) PCT Filed: Oct. 13, 2023
(86) PCT No.: PCT/IB2023/060324

§ 371 Date: Apr. 10, 2025

Related U.S. Application Data
(60) Provisional application No. 63/415,922, filed on Oct. 13, 2022.

(51) Int. Cl. G01N 33/483; A61B 5/268; A61B 5/293

(52) U.S. Cl. CPC G01N 33/4836; A61B 5/268; A61B 5/293; A61B 2562/028; A61B 2562/046

(57) ABSTRACT

Provided herein are electrodes having at least one grafted neuronal cell configured to electrically connect to a target tissue via the grafted neuronal cell. Also provided are cellular electrode arrays and methods related to the electrode and cellular electrode arrays. The array has a flexible polymer substrate with a top surface and an array of wells disposed in the flexible polymer substrate. The wells are configured to receive a neuronal cell and provide directed growth of sprouted axons and dendrites from the neuronal cell in a direction toward the top of the well. In this manner, the cellular electrode array can have high specificity, including for electrical interfacing, via electrical contact between the neuronal cells in each well and corresponding biological tissue and specific biological cells. The array configuration provides the ability to electrically interface with target tissue generally aligned with the array of wells layout.

### CROSS-REFERENCE TO RELATED APPLICATIONS

This application claims the benefit of U.S. Provisional Patent Application No. 63/415,922, filed on Oct. 13, 2022, which is hereby incorporated by reference to the extent not inconsistent herewith.

### BACKGROUND OF INVENTION

Provided herein are devices and related methods for electrically interfacing with biological tissue in a reliable and highly specific manner, thereby achieving high spatial resolution including for implantable applications. The devices have a micro-electrode array configuration with grafted cells reliably positioned within the array that are capable of electrical connection with tissue adjacent to the device.

Although the devices provided herein share some structural similarities with organ-on-a-chip devices, there are crucial difference in intended purposes. While organ-on-a-chip devices incorporate living cells to achieve in-vitro simulation of physiology, those devices are not intended for and have important differences with a device the electrically interface with biological tissue in a highly specific manner. For example, EP Pat. Pub. 2730645A2 titled “Organ-on-a-chip-device” is to establish or maintain organs or organoids as well as various cell niches.

Similar differences are found for lab on chip testing devices that collect electric signals, such as U.S. Pat. No. 7,395,474 titled “Lab-on-chip system and method and apparatus for manufacturing and operating same.” Such lab-on-a-chips usually do not, however, contain grafted cells that are capable of specifically electrically interfacing with adjacent tissue in a manner that provides relatively high spatial electrical interfacing resolution and they cannot stimulate or send encoded signals to the tissue.

A number of experimental MEMS have been developed by academic labs to study guided axon growth [1], [2] Those devices, however, are not neural interfaces and do not address the fundamental function provided with the instant devices and methods, namely forming highly specific electrical connections between biological tissue, including neural tissue, and electronic devices. Accordingly, there is a need in the art for devices wherein each unique electrical contact from the device corresponds to fewer corresponding electrical interconnects with the associate biological tissue. In this manner, the device is characterized as having high specificity, with the ability to electrically interface down toward the single or a few biological cells in the to-be-interfaced tissue, with corresponding functional advantages related to control/measure of smaller sub-sections of the biological tissue. Functionally, this provides a finer detail within the entire footprint of electrical contact.

Electrode arrays are a critical physical component of electrical interfaces with biological tissue that allows researchers to collect electric activity from nervous systems and stimulate activity in them. Three main types of commercially-available electrode arrays are: Surface Arrays (NeuroNexus), Utah Electrode Array (The University of Utah), Wire (Neuralink Corp.). All those devices, however, have poor specificity. This arises from the nature of electric fields in conductive medium, which spread by ionic gradient and the fact that electric field strength declines approximately proportionally with the distance. [3]

This electric field spread by neuronal cells in an ionic medium means that an extracellular electrode will tend to pick up activity from a large number of cells (hundreds to thousands). This is called a local field potential and it is difficult to isolate activity of specific cells from the recording because many cells are active at the same time. This means that electrical detection and/or actuation is difficult to isolate to any two cells with overlapping arborization or cellular processes. Similarly, there is a need to ensure electrical contact is in well-defined regions having a small footprint, thereby ensuring there is a small subset of cells that can physically and electrically interact with the device, including an area that is less than 100 μm², 10 μm² or 1 μm². Stimulation is equally problematic, as a pulse at an electrode could activate dozens to hundreds of cells in a diffuse manner. This occurs regardless of manufacturing precision. Although microfabrication techniques can produce contacts 10 nm apart, those contacts continue to individually pick up activity from hundreds to thousands of cells. For example, the density of neurons on the surface of rat visual cortex is estimated to be in the range of 70,000/mm³ [4] and electric field from each cell can spread over hundreds of microns.

Conventional devices include those that rely on indirect methods, such as thermal-optical stimulation [5], ultrasonic stimulation [6], and trans-cranial magnetic stimulation [7] attempts to trigger activity in neurons through intervening tissues. The precision of those devices, however, are inherently limited because: (1) the skull is a very good low-pass filter. (2) Relative biological motion between energy source and target tissue. (3) Many of them rely on slow actuation mechanism like thermal energy transfer. In addition, those devices are limited in that they cannot record simultaneously while stimulating. The second and the last point also apply to infrared thermal-optical stimulation even through it can to be relatively precise when applied directly to the brain via optic fibers.

Techniques such as EEG and Functional MRI (fMRI) attempt to record neural activity through intervening tissues. Resolution tend to be comparatively poor: EEG uses large macro electrodes that sample activity over entire section of the skull. fMRI has lower resolution than normal MRI, with spatial resolution of 0.75-0.5 mm achieved only in extremely strong magnetic fields [8], [9]. Temporal resolution is in the order of seconds. In addition, fMRI requires the subject to be stationary and so is not compatible with behavior experiments. Neither of those techniques can be used for stimulation.

There are also imagining techniques that can use retroviruses to deliver florescence genes to neurons or employ genetically engineered animals with florescence neuron [10]. A transparent window is installed on top of the skull for microscope imaging. The resolution and accuracy tend to be relatively good, but those methods require the animal to remain still and, therefore, are less useful for behavioral experiments. In addition, those methods cannot be used for stimulation without using additional complex optical caging techniques to make neurons sensitive to light.

Electrocorticography (EcoG) is similar to EEG but places electrodes on the surface of the brain instead of the top of the scalp. It's often used to locate focus of epileptic seizures. ECOG usually use macro electrodes, unless a flexible array is used (see Flexible Surface Array). Resolution is higher than EEG, but still relatively low and more suited for “bulk” interfacing, especially when compared to the micro-electrode arrays described and claimed herein.

Another conventional device is a glass micro-electrode array, such as a Silver/Silver Chloride (Ag/AgCl) electrode and/or gold or titanium contacts built onto a glass substrate by surface micro-machining (Multi Channel Systems MCS GmbH (a divisional of Harvard Bioscience, Inc.). Those devices are used only for in vitro studies as their size and rigidity make them impractical as an implantable devices.

Other devices include flexible surface arrays that combine characteristics of ECOG and Glass electrode. They are generally a grid of micro-electrode (usually PEDOT: PSS) built onto the surface of a Parylene film by surface micro-machining. Surface electrodes are less rigid then penetrating arrays (see Utah Array), and thus develop less mechanical problems. Those devices are usually placed on the surface of the brain, and have relatively good resolution. However, the specificity remains poor because electric field from multiple neurons overlap. Accordingly, there remains a problem in the art with respect to achieving specificity (e.g., a more one-to-one device to biological cell electrical interconnection), regardless of the underlying manufacturing precision or resolution.

The Utah Array, first developed by University of Utah, is comprised of grids of penetrating microneedles on top of a rigid silicon substrate which contains electrical interconnects. Each microneedle contains multiple Ag/AgCl electrodes along its length. Those electrodes are good at sampling signals from deep layers of the brain. They tend to have high precision, and can sometimes achieve good specificity. For example, it is possible to slowly drive a Utah electrode into the brain while listening for spike trains recorded when a needle penetrate into a cell. While excellent for acute studies, Utah electrodes cause neural tissue damage, including over the long term. The brain is in constant biological motion, including due to breathing, body movement, respiration and blood flow. Because the Utah array is rigid, the relative mechanical differences causes constant force mismatch and abrasion around the array. This in turn triggers tissue reaction, resulting in the electrode being gradually encased in layers of scar tissue, increasing impedance and eventually rendering the device useless.

Microwire based devices utilize wire electrodes formed from electrically-insulated microscopic silver wires with insulation stripped at the tip. They are individually inserted into the brain either manually or by a robotic device (Neuralink Corp., Fremont, CA). While soft at the macroscopic level, those wires are nevertheless rigid enough to penetrate small distances. Both resolution and precision are limited with the manual method, because it is physically challenging to control and maneuver wires precisely, including in a biological tissue. This may have been improved by robotic devices since, given the presentation by Neuralink Corp.

However, it is simply not realistic for a robotic surgical tool to reliably target single neurons; therefore, robotic surgery does not solve the fundamental issue of specificity: wire electrodes will still sample overlapping local field potential regardless where they are inserted. Furthermore, micro-wires are extremely fragile and break whenever subjects undergo rigorous movement. While less so than Utah Arrays, impedance tests and histologist analysis also reveal scar tissue build-up near the electrode. Often a large current must be applied to burn through the scar tissue, which is an accumulative risk to subjects in long-term studies.

Examples of existing electrode interfaces include the Utah array (see, e.g., www.sci.utah.edu/~gk/abstracts/bisti03/), surface/flexible array (see, e.g., www.multichannelsystems.com/products/flexmea36), and glass array (see, e.g., www.multichannelsystems.com/products/60pedotmea20030ir-au-gr).

From the above, there is a need in the art to address the problem of the electric field spread and corresponding degradation in device functionality with respect to electrical interfacing with biological tissue. That problem is addressed herein by providing devices having a high specificity with respect to electrical interfacing with biological tissue, with up to a single or at least very small number of biological cells that electrically interface with each potential electrode contact of the devices provided herein.

### SUMMARY OF THE INVENTION

The specificity problem described above is addressed herein via a novel micro-fabricated electrode array which uses living cells to form contacts, and more specifically electrical contacts including synaptic contacts, between conducting polymer electrodes and target biological tissue, including neural tissue. Through that physical contact, specific electrical interfacing is achieved. The devices provided herein comprise a flexible micro fabricated device made from a polymer, such as PDMS (polydimethylsiloxane) punctuated by a regular grid of wells. The polymer may be on the order of 1 mm thick, such as between about 100 μm and 2 mm thick, depending on the operating conditions and application of interest, including the electric field spread into the well. Conductive polymer contacts are positioned within a well on a surface location, including located at the bottom of the wells. The other top end of the well is open. Neurons are introduced into the wells and cultured, including with hydro-gels, to guide sprouted axons and dendrites towards the open end of the wells. Those sprouted axons and dendrites are available to make physical contact with target neural tissue. The combination of the precisely located sprouted axons, their one-to-one contact and electrical interconnection with biological tissue, and the special electrical grounding of the device surface facing the biological tissue, provides the device with the benefit of high specificity in combination with high resolution and attendant functional benefit, including: the ability to precisely, reliably and continuously electrically interface with biological tissue without damage or trauma, including down to a very small sub-regions, with each sub-region specific to an underlying electrical connection defined by a single well.

More generally, provided are electrodes with grafted neuronal cells configured to establish a biological electrical connection between the grafted neuronal cell and a target neuronal cell in a biological tissue in which the electrode is positioned. In this manner, substantially all electrical connection between the electrode and the biological tissue is through the biological electrical connection. The electrically conducting material of the electrode has an outer surface, wherein the outer surface is optionally formed of an outer material that is different from a core material of the electrically conducting material, with at least one neuronal cell grafted to the outer surface of the electrically conducting material.

Provided herein are cellular electrodes, including in the form of cellular electrode arrays, and related methods of making and methods of using any of the cellular electrode arrays to electrically interface with biological tissue and for making any of the cellular electrode arrays. For example, provided is a cellular electrode array comprising a flexible polymer substrate having a top surface and an array of wells disposed in the flexible polymer substrate. Each well has an inner-facing surface that defines a well volume. The inner facing surface may comprise a bottom surface and side wall surfaces that extend from the bottom surface to the flexible polymer substrate top surface. A conductive contact, such as a conductive polymer contact or a AgCl contact, at least partially covers the well inner-facing surface, such as positioned at the bottom surface and/or around at least a portion of the side wall. Preferably, the conductive contact is a conductive polymer contact, although other electrically-conductive materials are compatible with the electrode arrays, particularly materials that are flexible and are not rigid, so as to not adversely impact the overall device conformability to the biological tissue. In this manner, a well depth corresponding to a distance between the flexible polymer substrate top surface and the well bottom surface is defined. The well volume may be characterized in terms of the volume, and for a circular well cross-section the volume may be calculated as Hπ² (equation (1)), with H the well depth and r the effective radius of the well, such as d/2, with d the effective diameter of the well. Of course, the invention is compatible with any of a range of well cross-sections, including non-circular cross-sections such as ovals, rectangular, square and the like. Wells may have shapes with non-constant cross sections, such as tapers and funnels, including so as to receive a droplet. Depending on the application of interest, the well volume can be between 1 μL and 2 mL, including between 10 μL and 1 mL. The well may be configured to contain a hydrogel in each well and to receive a neuronal cell. In this manner, the hydrogel facilitates directed growth of sprouted axons and dendrites from the neuronal cell in a direction toward the flexible polymer substrate top surface. The ends of the sprouted axons and dendrites are available to physically contact biological cells, including biological cells that are electrically active, generating and/or responding to electric stimuli or field.

The conductive polymer contact may be positioned at the well bottom surface and/or along sidewalls and portions thereof.

The polymer substrate that is flexible facilitates conformal contact with a biological tissue, including a biological tissue that is curved or has a time-varying surface shape. In this manner, the cellular electrode is configured to operably connect flush against a tissue surface, including a tissue that corresponds to a brain surface with the operably connected mediated by synaptic connections between the neuronal cells in the wells (e.g., “grafted neuronal cell) and dendrites in the biological tissue, including dendrites from brain adjacent to the brain surface for biological tissue that is brain. Accordingly, “grafted” in the instant context refers to the neuronal cell that is reliably supported by wells such that there is sufficient proximity to an electrode surface that there is the ability of each of the grafted neuron and the electrode to electrically influence each other's electric field.

Each well may have a well depth that is between 10 μm and 2 mm.

Each well may have a volume that is between 10 μL and 1 mL.

Each well may have a characteristic diameter of between 1 μm and 1 mm. In other words, the volume of the well may be converted into a characteristic diameter if the well depth is known via equation (1).

The cellular electrode array may have a well density of between 0.001 wells/μm² (1000 wells/mm²) to 0.1 wells/μm² (10⁵ wells/mm²).

Adjacent wells may be separated by a separation distance of between 10 μm and 1 mm. In this manner, the resolution of the device may be controlled via well density and/or well spacing, depending on the application of interest. The specific connection between the grafted cells in the wells to the corresponding tissue provides the high specificity. The well density may be constant or may spatially vary, with certain sub-regions of the device having a higher resolution than others. For example, the central 50%-75% of the device may have a higher well density than the outer perimeter area of the device.

The polymer substrate may be conformable, so that it conforms to an irregularly-shaped tissue surface without adversely impacting device functionality. The conformability may be described in terms of macroscopic and/or microscopic adaptations to the curvature of the tissue surface, with the adaptations ensuring there can be reliable electrical connection between the grafted cell and the biological tissue. This conformability can be important because if the separation distance is too great, there simply will be too great a distance to span for electrical connections to be established and/or maintained.

The conductive polymer may have a range of thicknesses and/or a range of layer numbers. For example, there may be a grounding layer and/or electrical contact layer. The layer thickness may be between 1 μm and 100 μm. Optionally, the conductive polymer layer has a thickness that is less than the substrate thickness or well depth.

The non-conductive PDMS layer on the conductive PDMS layer forms an insulated conductive layer that is an electrical interconnect between the electrodes and external electronic components, such as amplifiers, cables, or PCBs, and to controllers, computers, and the like. Of course, external electronic components is used broadly herein. They also include, but are not limited to, contact pads on the other side of the array which in turn connect to other external electronic components (e.g., to facilitate reading a signal out of the array). On-board amplifiers may also be characterized as an external electronic component.

The conductive PDMS layer can be a PDMS composite layer. The electrical connection with the grafted neuronal cell need not be a physical connection, but instead the grafted neuronal cell, due to its proximity with the electrode (including electrically-active material such as conductive contact), facilitates the ability to detect an electric field projected by the grafted neuron, and similarly influence the electric field around the grafted neuron.

The flexible polymer substrate may be formed of polydimethylsiloxane (PDMS), including layers of PDMS for insulative and/or electrical conductance properties. A conducting layer of PDMS may be formed from a composite, such as PDMS spiked with an electrically conductive material to ensure current may travel through the PDMS composite conductive layer. For example, there may be at least one conductive PDMS layer and at least one non-conductive PDMS layer.

The non-conductive PDMS layer may comprise a spin coated layer on the conductive layer to form an insulation layer having an interconnect passage for receiving an interconnect to electrically contact for the conductive PDMS layer.

The cellular electrode array may comprise at least two non-conductive PDMS layers, including at least three, and may have any of a range of conductive and non-conductive PDMS layers.

The cellular electrode array may further comprise neuronal cells positioned in the wells, including grafted onto the conductive polymer contact. Microfluidics may be employed to reliably deposit a desired number of cells into each well, such as ranging from between 1 and 10 cells, 1 and 5 cells, and about 1 cell per well.

The neuronal cells may be selected to synaptically connect to a subject during use, and may further be selected to be immune compatible with a subject for in vivo use.

Any of the cellular electrode arrays may have a top surface of the flexible polymer that is functionally an electrical conductor so as to ground the cellular electrode array. In this manner, during use electrical communication between the cellular electrode array and a subject is confined to the neuronal cells in the wells synaptically connected to the subject (in vivo) or to a volume of an explanted tissue (in vitro or ex vivo).

Alternatively, instead of a simple grounding layer covering the entirety of the top surface, the grounding layer cover only part of the top surface. A conventional surface having a micro-electrode array may be provided onto the part of the remaining top surface for simultaneous collection non-specific electric field activity. This configuration can be useful for debugging or to serve as a backup.

The cellular electrode array may have a device footprint of up to 1000 mm², and optionally with up to 10,000 unique contacts between the neuronal cells in the wells and a tissue in which the cellular electrode array is implanted. The cellular electrode arrays provided herein are compatible with any of a range of footprint sizes, depending on the application of interest, along with any number of wells with a range of well density. One of ordinary skill in the art can select the appropriate well density based on the desired degree of actuation control and/or measurement resolution.

Also provided herein are methods of electrically interfacing with biological tissue using any of the electrodes or cellular electrode arrays described herein. The method may comprise the steps of: grafting a neuronal cell in at least a portion of, or each of, the wells of the cellular electrode array (or to an electrode outer surface for the more general electrode configuration). The cellular electrode array (or the electrode for the more general electrode configuration) with the grafted neuronal cells is contacted with the biological tissue. For example, the electrode array may be positioned such that the top surface of the substrate contacts the biological tissue so that the sprouted dendrites and/or axons that extend out toward or past the well can also contact the biological tissue. In this manner, at least a portion of the grafted neuronal cells are electrically connected to biological cells adjacent to the grafted neuronal cells, thereby forming electrical contacts between the biological tissue and the cellular electrode array. The biological tissue can then be electrically interfaced via the grafted neuronal cells that are in electrical contact with the biological tissue. Similar interactions occurs for the more general electrode configuration.

The electrical interfacing may comprise electrically actuating the biological tissue and/or measuring an electrical parameter of the biological tissue.

The electrically interfacing step may comprise measuring current flowing to or from at least a portion of the grafted neuronal cells that are electrically connected to biological tissue. The electrical interfacing may comprise generating a map of electric potential over the interfaced area, corresponding to a portion or all of the electrode array footprint. Such a map, because of the high specificity of electrical interfacing between biological tissue and the array via the grafted neurons, will have very high spatial resolution with the measured electric potential measured at each well position of high accuracy and sensitivity as the measured electric potential is specific to that location with minimal cross-talk from non-adjacent regions of biological tissue. The interfacing may also utilize statistical methods for teasing out the measured action potentials from a bulk signal being detected by the electrode through a path independent of the grafted cell.

The electrical interfacing step may comprise, as desired, collection of bulk signals mixed with specific signals, such as signals via the biological electrical connection, such as action potentials. Subsequently instead of discarding such bulk signals as noise, statistically separating the bulk from the specific signals, using techniques such as PCA, ICA, frequency domain filtering or any other technique that exploit the characteristics of action potentials or spike trains.

The method may further comprise the steps of: selecting a portion of the wells having neuronal cells electrically connected to biological cells and electrically interfacing the selected portion of the wells with the biological cells. The selected portion of wells may correspond to a spatial region having a surface area of between 1 μm² and 5 mm².

The method may further comprise the step of matching a mechanical property of the cellular electrode array with a mechanical property of the biological tissue. The mechanical property may be a modulus, such as a Young's modulus, that is within 50%, 20% or 10% of each other.

The arrays provided herein are compatible with any of a range of biological tissue, including biological tissue where electric potential and electrical activation are of importance. The biological tissue may be selected from the group consisting of: brain, spinal cord, peripheral nerves, a three-dimensional cell culture (including a bioartificial organ), and organotypic cultures. Particularly useful is brain tissue, where the brain tissue comprises neuronal cells with brain dendrites that are capable of synaptically connecting to the grafted neuronal cells of the array, thereby providing the specific and reliable electrical connection between the electrodes of the array and the brain tissue.

Also provided herein are methods of making a cellular electrode array. The method may comprise the steps of: forming a recessed master comprising an array of relief features on a silicon wafer surface or SU8. The relief features may correspond to the desired array of wells size, location and geometry. A polymer layer is cast against the array of relief features and the cast polymer layer cured. The cured polymer layer is patterned to expose a top portion of the relief features. Electrode contacts are deposited onto the exposed top portion of the relief features and an insulating layer deposited over the deposited electrode contacts. The electrode contacts are electrically interconnected for electrical connection to a to an electrical component, including one or more of an amplifier, a printed circuit board (PCB), a transmitter, an analog filter, and/or an analog to digital converter (ADC) or any component useful for connecting or communicating directly or indirectly with an electronic device, such as a computer. The silicon wafer is removed from the cast polymer and other layers, thereby making the cellular electrode array having an array of wells, with the wells corresponding to the array of relief features.

The patterning may comprise removal (e.g., etching) and/or addition (e.g., deposition) including by selective curing.

The forming the recessed master may comprises processing the master silicon wafer surface by: reactive ion etching; and/or cutting with a die saw.

The casting step may comprise: casting a main body layer of PDMS to cover the exposed relief features on the master, wherein the main body layer of PDMS is not electrically conductive. Conductive paths are patterned onto the main body in conductive PDMS composite by etching, lift-off or selective curing. Grounding layer can be created separately by spin coating on another wafer and attached to the main body by adhesion or transfer printing after the main body is removed from the master.

The method may further comprise the step of re-using the recessed master to make another cellular electrode array. In this manner, cost savings may be realized.

The method may further comprise the steps of: providing neuronal cells to the wells; providing a hydrogel, including a collagen solution, to the wells; culturing the neuronal cells to generate axon and dendrite growth. In this manner, the array is ready for contact with a biological tissue. Accordingly, the method may further comprise the step of contacting the cellular electrode array with a biological tissue so that the neuronal cells electrically interface with the biological tissue, including biological cells within the biological tissue. The method may be practiced on the living body, including a human or a non-human animal. The method may be in vivo, in vitro or ex vivo.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIGS. 1A-1C: Schematic illustration of the cellular electrode array, with a side view (FIG. 1A), a top view (FIG. 1B) and an implanted view with attendant electronics for measuring a signal output (FIG. 1C).

FIGS. 2A-2E: manufacturing steps for an electrode-neuron grafted array.

FIG. 3 is a flow-chart schematic of certain relevant manufacturing steps for making the electrode array. Dark solid is SU8; light solid is PDMS; left hatching represents a conductive composite; right hatching represent wells and holes; horizontal hatching represents metallization; vertical hatching represents channels.

FIG. 4 is similar to FIG. 3, but provides another embodiment of selected manufacturing steps for making the electrode array.

FIG. 5 is a schematic of the electrode in use to form a biological electrical connection with a target cell via a grafted neuronal cell.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

“Interfacing” is used herein to refer to the ability of two materials or components to electrically interact with each other, so that a change in an electrical parameter (e.g., potential) of one material or component affects the state or status of another material or component, without adversely impacting the functionality of each material or component. Interfacing is used broadly herein to include electrical stimulation (e.g., “actuation”) and/or electric parameter detection (e.g., “measurement” or “detection”). For example, the arrays provided herein may electrically stimulate biological cells and/or detect an electrical field or electrical parameter (potential, current) generated by biological cells.

“Flexible” refers to the ability of a material, structure, device or device component to be deformed into a curved shape without undergoing a transformation that introduces significant force that would otherwise result in failure. A flexible material, structure, or device may be deformed into a curved shape without introducing a force greater than the failure point of the material.

“Conductive polymer composite” refers to a polymer that is capable of conducting an electric charge. The conductive polymer contact may be formed from a composite material, including a mixture of several polymers and a mixture of polymer with distributed metallic elements.

“Operably connected” or “in operable connection” refers to a configuration of elements, wherein an action or reaction of one element affects another element, but in a manner that preserves each element's functionality. For example, a cellular electrode array in operable connection with the biological tissue or surface refers to the ability of the electrode array to electrically interface with the tissue, including cells, without adversely impacting the tissue or the array.

“Electrode” refers to the component of the cellular electrode array that can be electrically energized to electrically stimulate a biological material and/or to detect an electrical parameter arising from the biological material. The electrode may be a conducting metal. The electrode may be a composite, with a core material that is covered or coated by an outer material that is different from the core material. The outer material may correspond to coatings of active sites where electrical connection is desired. As but one example, the electrode may be coated in Poly(ethylenedioxythiophene) (PEDOT). The electrode may be configured to graft to a neuronal cell, so that substantially all of the electrical connection with a biological tissue is via the grafted neuronal cell(s). In this context “substantially all” refers to at least 80%, at least 90%, or at least 95% of electrical current that travels between the biological tissue and the electrode via the grafted neuronal cell. “Substantially all” also refers to the situation where electrical signals in the form of action potentials are provided between the electrode and the biological tissue without loss or substantial degradation of the action potentials, including the frequency of action potentials.

“Device footprint” refers to the surface area defined by the perimeter of the polymer substrate.

“Adjacent” refers to biological tissue that is sufficiently close to the grafted cells in the wells of the electrode array is able to make electrical contact with the tissue, including biological cells of the tissue. The term is intended to reflect that there is a constrained region of contact, as the grafted cells have a spatial limit as to how far from the well in which they are contained can establish direct electrical contact. At least part of the constraint is the distance from the well the sprouted dendrite or axon can grow. Accordingly, adjacent includes a functional definition based on typical maximum axon length is less than 100 μm, including between about 20 μm and 60 μm. In one aspect, any tissue that is within 100 μm from a reference location, such as an edge of a well, is considered to be “adjacent” to that reference location.

“Bulk electrical interfacing” refers to an electrical interface with electrodes on the surface of the device, where an electrical parameter (e.g., electrical potential) or electrical signal (e.g., current or potential) is measured or provided directly with the electrodes without the biological/synaptic connection.

“Well” is used broadly herein to refer to a volume in a flexible polymer substrate that can reliably support growth of a biological cell under culture conditions. The well may have any of a range of shapes. For example, the cross-sectional shape may be uniform, such as cylindrical in nature. Alternatively, the wall may have a funnel or taper shape, with a larger cross-sectional area toward the flexible polymer substrate top surface, tapering to a minimum cross-sectional area in a direction away from the top surface. The well can be shaped depending on the application of interest. For example, to receive a droplet containing very few neuronal cells (e.g., down to about 1 neuronal cell/droplet), the well may have a tapered configuration to help guide and confine the droplet to the well.

The invention can be further understood by the following non-limiting examples.

### Example 1: Device Overview

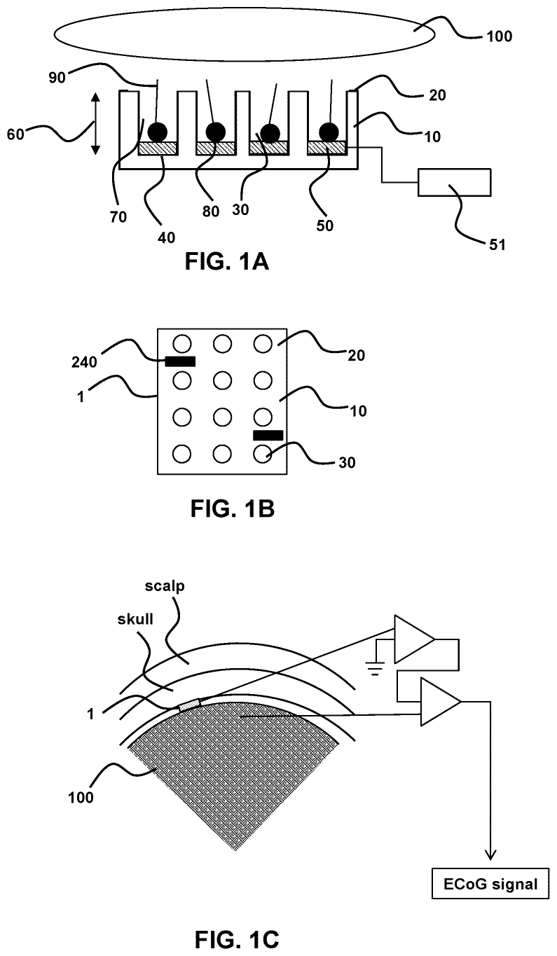

Referring to FIGS. 1A-1C, a cellular electrode array 1 is formed from a flexible polymer substrate 10 having a top surface 20 and an array of wells 30. Each well has an inner facing surface 40, including a bottom surface, and a conductive polymer contact 50 (illustrated as positioned to form the bottom surface). Together, a non-conductive PDMS layer and a conductive polymer layer can effectively form an insulated conductive layer that is functionally an electrical interconnect between the conductive polymer contact 50 and an external electronic component 51. Well depth is illustrated by arrow 60. A hydrogel 70 can be disposed in each well. For simplicity, one neuronal 80 with sprouted axon and dendrite 90 is illustrated per each well, available for contact with a biological tissue 100. FIG. 1A is a side cross-section and FIG. 1B a top view that also illustrates conductive top surface 20. FIG. 1C illustrates the cellular electrode array 1 implanted under the skull and electrically interfacing with biological tissue that is brain. For simplicity, the interface is shown in the direction of detecting a signal. Of course, the signal may be generated by the device and used to actuate biological tissue. Both the detecting and generated signal may be spatially varying over the footprint area (see, e.g., FIG. 1B showing the footprint of a 4×3 electrode array).

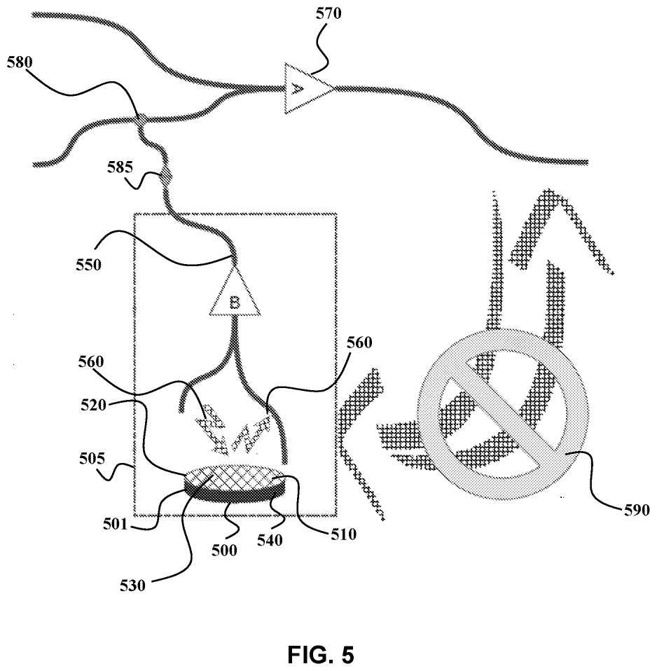

Referring to FIG. 5, an electrode 500 (which can, functionally, be equivalent to conductive polymer contact 50 of FIG. 1A-1C, including by being formed of a plurality of PDMS layers 501) formed of an electrically conducting material 510 having an outer surface 520. The electrode may be formed of a plurality of distinct materials, such as an outer material 530 that is different from a core material 540 (e.g., the inner body) of the electrode. Line 505 represents system boundary, such as the implanted portion footprint. A neuronal cell 550 is electrically connected to the electrode, as represented by the two-way electrical current arrows 560. In this manner, neuronal cell 550 is characterized as a “grafted” neuronal cell with respect to the electrode 500. With respect to a target neuronal cell 570 in a target or biological tissue, the grafted neuronal cell 550 has a biological electrical connection 580. This is reflected by the two-way arrows 585, indicating that the electrode may be used to stimulate target 570 or to detect an electrical parameter of target 570. Arrows with strike-through 590 corresponds to leakage of electric current between biological tissue and electrode, reflecting that substantially all electrical connection is between the biological electrical connection 580 with attendant arrows 560 rather than “non-specific” electrical connection of electrical path 590. For example, at least 80% of the electrical current may be via path 560 compared to the non-specific path 590.

### Example 2: Summary of Method of Making the Device

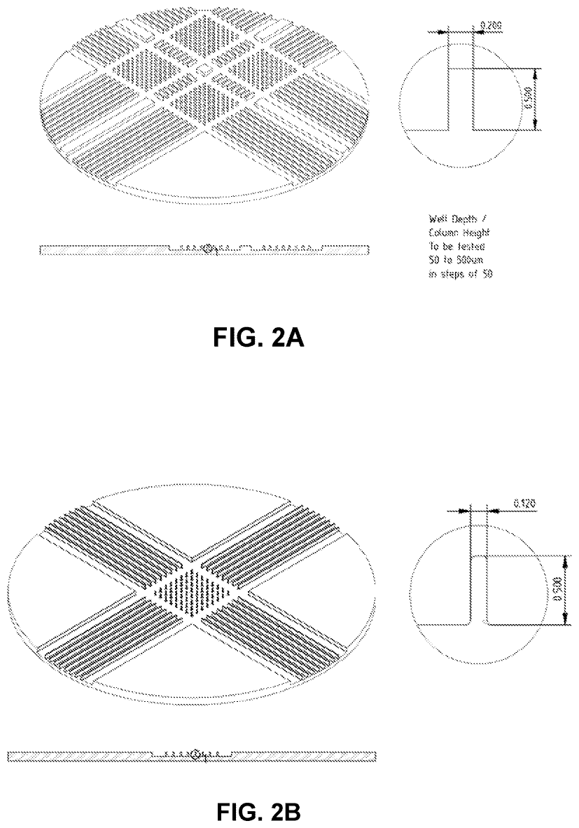

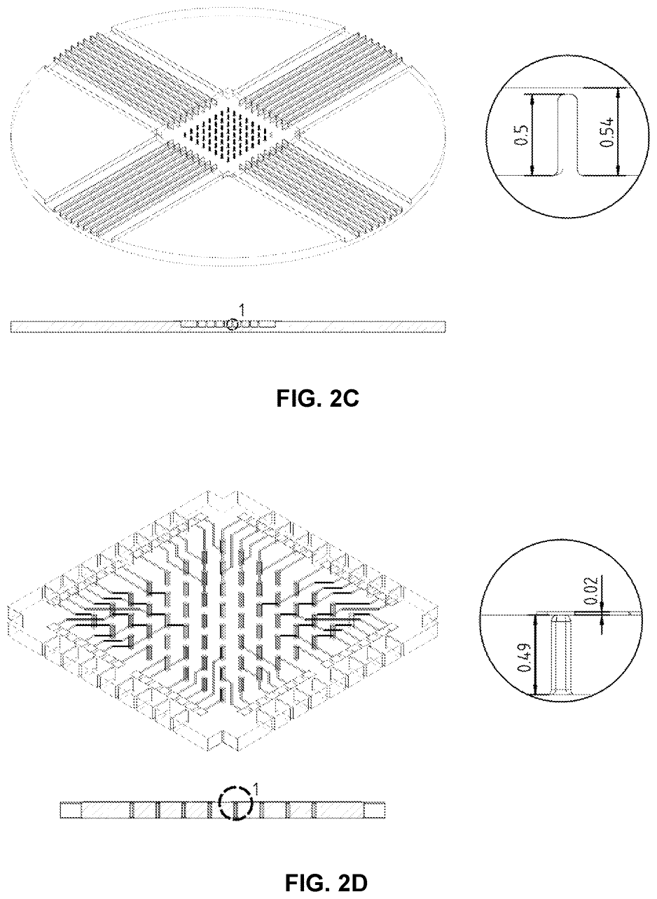

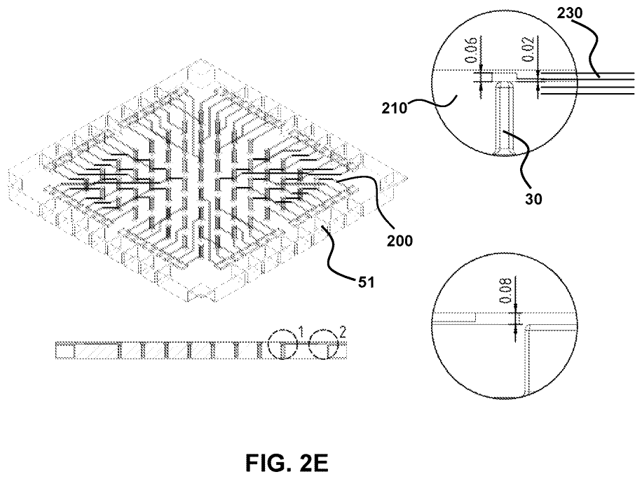

FIGS. 2A-2E are summaries of various steps that can be utilized to make an electrode-neuron grafted array. Unless noted otherwise, dimensions are only exemplary. FIG. 2A illustrates multiple potential devices on one wafer. For simplicity, subsequent figures illustrate one device. All units are in mm. The designs and dimensions shown are a representative embodiment. Step nos. refer to the steps further explained in Example 3.

FIG. 2A illustrates an initial step with intersecting grooves cut into the surface of the wafer with a diamond saw. An 8 by 8 well design is shown as an example, actual well density can be much higher.

FIG. 2B illustrates second step described in step 1.b)ii below, isotropic wet etching creates rounded profiles. The profile created using 1.b)i will be similar, except missing rounds near the apex and the bases of the columns. Either process is acceptable.

FIG. 2C illustrates casting of the Device Body described in step 3. The thin layer of conductive PDMS (10 μm in this example) serves as the ground and can be deposited before the main body or attached as the final step. The main body should exceed the top of the columns by a known amount.

FIG. 2D illustrates deposition of electrodes and part of the interconnects after steps 4.a), 4.b) and 4.c). Device Body should be etched to the top of the master, then conductive PDSM is patterned by selective curing (if photo-reactive) or by etching with a metallic mask layer (if not photo-reactive). Wiring is optimized by any number of techniques and processes, including but not limited to manual determination and/or using electronic design automation (EDA) software for large arrays. Note some electrodes are not connected to contact pads in this layer because multiple layers are used for dense arrays. The interconnect between electrodes and contacts can be created manually, using an auto-routing function of most EDA software.

FIG. 2E illustrates the completed electronics after step 4. Electrical interconnects 200 may not all fit in the same layer on a dense electrode. The interconnects 200 electrically connect the electrodes to the contact pads at or toward the device edges. Interconnects connecting to various regions of electrodes may be at different layers in the device. For example, the interconnects that electrically contact the electrodes positioned in the device middle region may be on a layer above the other interconnects. Accordingly, the devices can be built in layers 210 by repeating steps 4.b), 4.c), 4.d) and 4.e) in example 3. The pattern of external contact shown in this example is designed to be compatible with Multichannel System amplifiers for Glass MEA (alternative 3), described in 4.f) ii. Four layers are illustrated in the top inset figure, including top insulation. One layer is illustrated as having a thickness of 20 μm. The portions of the insets with the dimensions are illustrative electrode contact dimensions. Multiple layers 230 are schematically illustrated, and reflect that the interconnects and/or electrode contacts may span multiple layers.

### Example 3: Fabrication Method of the Micro Electrode Array

The devices provided herein can be implanted under the pia matter, flush against the brain's surface. When axons and dendrites from grafted neurons reach the opening in the well, they can synapse onto nearby dendrites from the neural tissue. The axons in electrical contact with the tissue can then conduct electric activity stimulated by the electrodes. Similarly, cells near the device will project axon collateral which synapses with dendrites in the wells, conducting signals to grafted cells. Because the device itself is dielectric (e.g., the device surface is effectively grounded so that signals cannot leak between cells and unwanted signals cannot leak from tissue to the device), the electric field cannot reach neighboring wells. Thus interference is limited by the number of cells in each well, a number controllable by device dimensions and cell culture parameters, such as plating density.

Various manufacturing processes may be employed to make an electrode array suitable for containing cells configured to electrically interface with a biological tissue.

One example of a method of making the device includes that summarized in FIGS. 2A-2E and as further explained below:
1. Use Utah array fabrication technique to create a recessed master with a regular array of circular columns. [13], [14]
   a. Starting with a 1,0,0 silicon wafer.
   b. Create high aspect-ratio profile of the columns using one of the two methods:
      i. Spin coat omi-coat and photo-resist. Pattern Photo-resist along the edge of the wafer, as well as in a grid pattern at intended location of wells. Use deep reactive ion etching to create an array of circular columns.
      ii. Use a diamond-edged die saw to cut regular pattern of grooves into the wafer. Repeat the cutting process from the tangential direction. These grooves will intersect to form a regular array of square columns. Subsequently apply isotropic wet etching to create circular profile of the columns as well as rounded bases.
2. Create a mixture of PDMS and conductive PDMS.
   a. PDMS mixture can be made or purchased as they are commercially available.
   b. PDMS degassed after mixing by centrifuge to remove bubbles. (https://blogs.rsc.org/chipsandtips/2010/08/17/degas-pdms-in-two-minutes/)
   c. One of these following Conductive PDMS solution can be made.
      i. EG, PEDOT: PSS PDMS, and Triton X-100. This mixture has practically the same mechanical property as PDMS, thus avoiding conductivity problems caused by micro-cracking arising from mechanical mismatch.
      ii. Commercial Photo-curing PDMS mixture. (Bluestar Silicones, Silcolease UV Poly 110) can be used as the basis for conductive mixture in part i [16]. Such a mixture can be patterned directly.
      iii. An alternative formulation is a mixture of PDMS, Silver, and 2-hydroxy-2-methylpropiophenone. This mixture is also photosensitive and can be patterned directly.
      iv. Carbon Black is interchangeable with silver particles for the mixture in part iii.
   d. Conductive PDMS should be degassed using the same method described in b).
3. Create the array of wells by pouring and casting PDMS. The electrode body can be created upside down.
   a. an initial grounding layer of conductive PDMS may be spin-coated onto the master. There will likely be a significant accumulation of solution at the rising edges away from the center of the wafer [20], this is not expected to cause an issue because the electrode is much wider than tall. A large tolerance in the thickness of the grounding layer is therefore acceptable. More preferably the grounding layer, however, is attached as a final step because a thin layer of conductive PDMS could coat the columns too and it may be desirable to avoid or minimize such a coating location. As provided below, to avoid this the grounding layer may be fabricated separately and attached by oxygen plasma bonding after detaching from the master.
   b. Apply vacuum to remove remaining gas bubbles and allow solution to settle for 30 minutes.
   c. Bake wafer at 150° C. for 2 hours to cure.
   d. Flow non-conductive PDMS solution into the master and precisely control the level of solution via one of the following methods:
      i. by precisely measuring the quantity of solution used. This volume can be calculated by weighting the master after step 1 to determine mass removed by bulk surface micromachining.
      ii. removing excess solution using a glass microscope slide with a round on the leading edge. The slide will move across the top of the master with a weight applied.
      iii. silanize a glass or silicon wafer with MPTS: (3-mercaptopropyl)triethoxysilane using standard techniques [53]. Hold the master (either PDMS or SU8) against the treated wafer under pressure (such as by clamping), flow PDMS mixture between them and cure under pressure. Peel off the wafer after curing the PDMS.
   e. Repeat step c. to cure.
4. Pattern the electrode contracts in Conductive PDMS
   a. Without removing PDMS dies from silicon master, use Reactive Ion Etching (CF4/O2) to etch the PDMS until top of silicon master is exposed. This will also etch small amount of the master, but PDMS will etch at ten times the rate of Silicon, and therefore should not be a problem. Doing this without peeling off the PDMS prevents alignment problems caused by shrinkage of PDMS material.
   b. A layer of conductive PDMS should be spin-coated on the top surface. Refer to step 3.a., 3.b., and 3.c. At this point, the top surface of the master-die complex should be more or less flat. Thus much less accumulation is expected to occur [19], [23]. The minimum thickness of spin-coated thin PDMS layer can be approximately 20 microns, including between 15 μm and 50 μm, and any sub-ranges thereof.
   c. Conductive PDMS can be patterned in one of two ways, depending on if a photo-reactive PDMS mixture from step 2.c. is used. Note that if formulation 2ci is used, water vapor may form bubbles inside enclosed spaces, and so may be cured at room temperature.
      i. If the PDMS mixture is not photoreactive, Conductive PDMS is patterned using a metallic photo-resist following a reactive oxygen surface treatment. It may not be possible to lower photomasks all the way to the die surface, but proximity printing is acceptable at tolerances involved. The exposed area can be etched by Reactive Ion Etching until top surface of non-conductive PDMS layer. A small amount of over-etching ensure no conductive material remains in the exposed area.
      ii. A photo-reactive PDMS mixture 2.c.ii. or 2.c.iii. can be exposed to UV and selectively developed like a photoresist.
   d. Non-Conductive PDMS can be spin coated on the top surface to create insulation between layers, repeat degassing and curing steps. Holes for interconnects between layers can be patterned onto the insulation or punched afterwards.
   e. Repeat step b., c., and d. once for each layer of electrical connections.
   f. electrical connections are different for different type of head-stage amplifiers. wire bounding techniques are IP of amplifier manufacturers and are only described for information.
      i. For ribbon connection such as TBSI of Multichannel flexMEA, A Parylene ribbon cable with conductive PEDOT: PSS can be manufactured as an additional layer. Device density on wafer must be reduced to accommodate this.
      ii. An interconnect glass PCB can be manufactured for compatibility with glass MEA amplifiers. The PCB is designed to compensate for the difference between the size of glass MEA and the instant micro device. Cellular Electrode Array is bonded to the PCB by reactive oxygen plasma surface treatment.
   g. Peel the completed polymer dies off wafers and cut them into devices.
   h. As desired, reuse the silicon master to reduce cost.

### Illustrated Alternative Fabrication Methods (for a Single Well)

This example is another embodiment related to Example 3, with manufacturing steps that can supplement or replace any of the steps provided in Example 3, as noted herein.

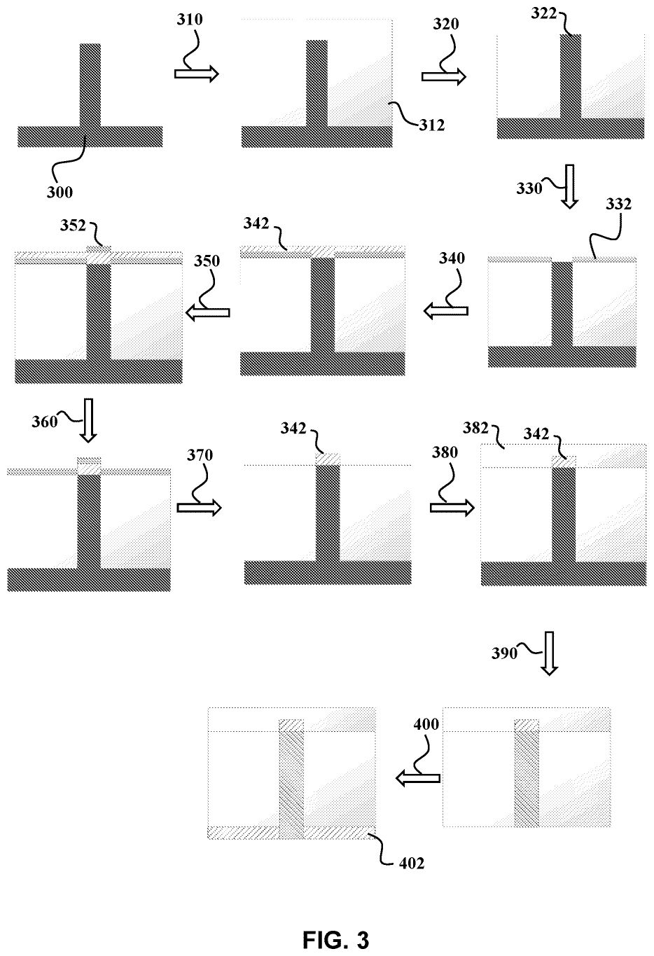

FIG. 3, schematically illustrates parts of a manufacturing process showing, for simplicity, a single well and its immediate vicinity, as a flow chart of steps. Of course, the process can be used for an array of wells and is compatible with any of a range of known processes in the art related to substrate micrometer-sized handling, deposition, etching, and equivalents thereto.

300 illustrates a SU-8 master comprising a pillar positioned at a corresponding well location. As noted, 300 is preferably an array of pillars corresponding to wells of the microarray. Optionally, the SU-8 master is functionalized, such as by silanization, to facilitate removal of the SU-8 master from another layer of material, such as a PDMS-based layer, including by peeling of the arrays from the SU-8 master.

In step 310, PDMS is cast over the master 300 to form a PDMS body 312, Preferably, PDMS body 312 is not conductive. In step 320, the PDMS body 312 is etched until the top surface 322 of the SU-8 pillars 300 are exposed.

The Conductive interconnects are subsequently patterned by applying any of a variety of techniques, such as surface machining process, so long as the techniques is compatible with PDMS-based materials. Conductive interconnects are formed from PDMS based conductive composite. The subsequent paragraph provides two specific examples of patterning: by masking and etching or by channel filling.

### Masking and Etching:

Pattern by masking and etching [21]: prepare positive and negative photomasks of each interconnect layer; optionally perform reactive oxygen treatment of the wafer; transfer negative photomask to aluminum hard mask on the top surface. For example, in step 330 a protective mask 332 is provided on the PDMS top surface and in step 340 a conductive path layer 342 is provided, such as by spin coating a PDMS based conductive composite. These may be: PDMS, PEDOT: PSS, Triton X-100 and ethylene glycol [15]; PDMS and carbon black [32]; PDMS and carbon nanotubes [37] [33]; PDMS and silver powder or silver nano wires [34].

In step 350 an etch mask 352 is provided to the positive photomask, such as an aluminum hard mask that is resistant to the PDSM etching of subsequent step 360, also generally referred to as an etch down step that removes PDMS-based material that is positioned over mask 332. PDMS etching is by any of a range of techniques known in the art, including: Dry Reactive Ion Etching (DRIE) using, for example CF4 and O2 or SF6 and O2; Wet etch using NMP (N-methyl pyrrolidinone) and TBAF (tetra-butyl ammonium fluoride). [36]; Combined wet/dry process, where dry etch by CF4 and O2 is followed by wet etch. [24]; Wet etch using concentrated sulfuric acid. [38].

After etching, the hard masks 332 and 352 are stripped revealing wire layer 342 over pillars 300. In step 380, insulation 382 is applied over conductive path layer 342, such as by an additional layer of PDMS by oxygen plasma treatment [39], [40]. In step 390 the arrays are detached and in step 400 bonded to a grounding layer 402.

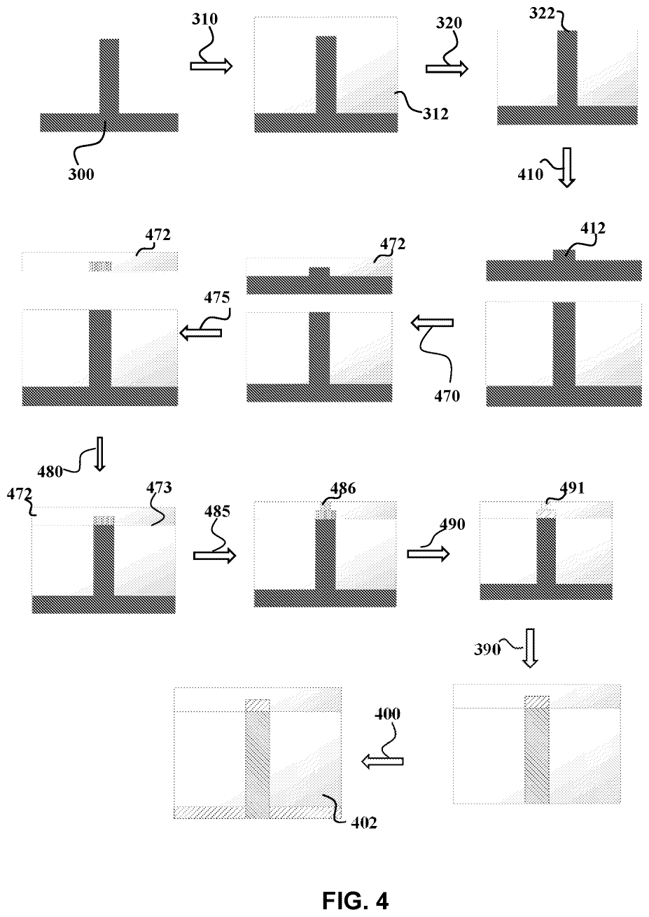

Channel Filling: An alternative to steps 330-380 of FIG. 3 is shown in FIG. 4, with initial steps 310 and 320 the same as in FIG. 3. In step 410 a new positive master 412 is provided and in step 470 a PDMS negative 472 of the interconnect layer is cast. The PDMS negative 472 is detached in step 475. In step 480 the PDMS negative 472 is bonded to the top surface 473 of the device body, such as by oxygen plasma treatment of both adjacent surfaces.

A punch hole 486 is made in step 485 to provide access via injection of a PDMS based conductive composite 491 and subsequent curing as illustrated in step 490. Note that the composite containing ethylene glycol will form bubbles when heated and should not be cured in an oven.

As in masking and etching, steps 390 and 400 are the final steps where the wells and the interconnects are bond to the separately created grounding layer. Conductive grounding layer and/or arrays for bulk interfacing are bond to the tissue-facing side by reactive oxygen treatment (see, e.g., step 400). Wafers are cut into devices and bonded to wires.

A particularly important aspect of the instant devices and methods is the ability to reliably add neuronal cells to the electronic device that is in the form of a microarray. Neuronal cells, in other words, can effectively be grafted to the device and ready to make an electrical connection with biological tissue. The electrical connection is fundamentally improved herein, in that each individual electrical connection between device (e.g., a neuronal cell in the well) and tissue is to as few as less than 10 cells in the tissue, less than 5 tissue cells, or as few as 1 tissue cell. The tissue cell may be in a tissue such as brain, spinal cord, or a peripheral nerve.

### Example 5: Grafting Neuronal Cells

With respect to obtaining and providing to the device an immunologically compatible cell configured to graft to the device and provide an electrical contact with a biological tissue, the following includes preferred embodiments for the methods of cell culturing:

1) Obtain immunologically compatible cells for the graft.
(a) Adult cortical neurons can be difficult to work with. However, for auto-graft, neurons can be obtained from the subject's own Dorsal Root Ganglion (DRG). They are routinely obtained as a part of thoracic vertebrectomy [42]. Neurons can be dissociated from the matrix by standard protocols.
(b) Neurons are immunologically privileged. Studies show that transplanted allo-graft neurons are not rejected by the immune system [43], [44]. Even Xenografts are known to survive [45]. This open up the door to use well understood Induced Pluripotent Stemcells (IPSC). Somatic cells such fibroblast can be transformed into IPSC by transient expression of transcription factors (for example using Oct3/4, Sox2, c-Myc, and Klf4) [46], [47]. IPSC linage are commercially available (for example ATCC's ACS1019).
(c) Subsequently, IPSC is induced to differentiate into neurons or precursors [48], for example with ASCLI, BRN2A, and MYTIL [25]. In addition, IPSC derived cells are commercially available (see, e.g., Elixirgen Scientific's QuickNeuron™ Series).
(d) Alternatively, for small animals, compatible neurons are obtained from an inbred rat strain, a bio-engineered twin or a cloned embryo.

2) Creating the culture medium following well established receipts. Either of serum-free or otherwise:
(a) Neurobasal w/B-27, 0.5 mM L-Glutamine, 1% heat-inactivated FBS, 2.5 g/L D-glucose, 20 ng/mL NGF, and 20 IM FdU+20 IM uridine. [49]
(b) Neurobasal medium (Invitrogen, Gaithersburg, MD) supplemented with 2% B-27 (Invitrogen), 1% penicillin-streptomycin (Invitrogen), and 0.4 mM of Lglutamine (Invitrogen) [50]

3) Transfer the PDMS devices to petri-dishes or culture flasks (see 5a) with wells facing up, and sterilize using one of the following techniques. Method a is preferred because it will also make the surface more hydrophilic. Note that gas sterilization is not recommended because gas will diffuse into PDMS and make it toxic.
(a) By Oxygen plasma treatment
(b) By autoclaving

4) PDMS is extremely hydrophobic, therefore action must be taken to improve surface adhesion, especially if step 3a is not taken. Cover the PDMS devices with culture media for several hours, to allow protein to absorb onto the surfaces. (a) Optionally, after covering the devices with culture media, transfer the dishes to a low vacuum, until bubbles in the wells dissolve into the media.

5) Plate cells at a density where, on average, there is one cell per well.

Optionally, transfer the PDMS device and cells to a flask and use a centrifuge adapter (Beckman Coulter SX4750) to force cells into the wells. Use PBS to wash away excess cells at the surface

6) Provide a scaffold for axon/dendrite growth. Both the following reagents are available commercially. Because these reagents are only liquid at low temperature, there is only a short time window to work with them. Therefore it is not advised to suspend cells in them, rather they should be applied in a separate step after cell-plating. Of course, the methods and devices provided herein are compatible with providing them to a surface before cell-plating.
(a) Collagen (e.g. Gibco™ A1048301)
(b) Becton Dickinson Matrigel™

7) Follow the instruction of reagent for gelling, for example, exposure to ammonia.

8) Introduce fresh media every 2-3 days. Allow 1-2 weeks for axon and dendrite growth and observe for their spread along the scaffold.

### Example 6: In-Vitro Testing

Resistive and capacitive leaks between wells without grafted cells may be tested without grafted cells. It is possible to test if stimulation artifacts appears in other channels without the cells. This test is especially important for dense arrays. The device surface facing the tissue is a solid conductive grounding layer, and so there should be no current flow between wells. This aspect is relevant for achieving high specificity between device and the tissue.

Because PDMS is optically transparent, the device can be directly visualized, including with a confocal microscope, by supporting the device with a microscope slide.

Because PDMS is soft, the device can be fixed in a dish and sectioned, such as sliced with a vibrotome, and stained (for example, with Nissil stain) for imaging, including by a transmission microscope. This aspect is useful for assessing if grafted cells are viable and living (live vs. dead stain).

Iterate design by simulation: (a) Stimulators apply bi-polar pulses. (b) This pulse introduces a voltage gradient between the grounding plate facing the tissue, and the electrodes at the bottom of the wells. (c) The amount of gradient required to trigger action potentials can be determined with in silico simulation, such as by NEURON software, and LFPy package in python. (d) Simulation and in-vitro results are used to inform device dimensions, such as well diameter, depth, geometry and spacing (density). (e) Likewise similar simulation can be used to optimize the array's ability to detect action potentials propagating inside the wells towards the cell body.

### Example 7: Cell Type Targeting

Targeting synaptic connections: (a) The type of cells grafted to the electrodes of the array influences the counter-part corresponding nearby cell (from the biological tissue) that forms a synaptic contact. This can be leveraged with targeted gene expression, to facilitate connection with only certain types of neurons in the brain. (b) This is a relevant consideration in view of the fact that the brain comprises different types of neurons in different brain regions.

The devices and methods are compatible with a range of cell types, including commercially-available cell types and custom-isolated cell types, so long as they can be provided at a low cell number to each well and are able to reliably secure electrical connection to a biological tissue.

For example, commercially available IPSC derived cells may be used to target specific type of neurons. Different types of neurons (for example Motor, Sensory, Cholinergic, Dopaminergic, GABAergic) preferentially form synapse with different type of cells. This can be used to communicate with specific type of cells in the vicinity of the array. Examples of commercially available cell types for use with the instant devices and methods include, but are not limited to, those provided in TABLE 1.

TABLE 1
Representative cell types (Cells derived from
Induced Pluripotent Stem Cells (IPSC))

| Cell type/ Vendor | AXOL | IXCells | Elixirgen |
|---|---|---|---|
| Motor | AX0078 | 40HU-006-2M | MT-SeV-CW50065 |
| Sensory | AX0555 | 40HU-013 | SS-mRNA-HC |
| Cholinergic | | | CH-SeV-HC |
| Dopaminergic | | 40HU-002 | DA-mRNA-HC |
| GABAergic | | 40HU-010 | GA-mRNA-HC\| |

### Example 8: Animal Surgery

1) Electrode implantation: Chronic Neural recording techniques are well established [52]. Procedures are conducted in compliance with sterile, anesthesiology, and animal ethics protocols.
(a) Prepare surgical tools as per sterile protocols.
(b) Place a rat under anesthesia using isoflurane or other agents, place the animal on a stereotaxic frame. Shave fur from its head. Make an incision on the skin with a scalpel and reflect over.
(c) Remove bone to make a window on the skull over the area of the cortex that is targeted using rongeurs.
(d) To surgically implant the electrode array, cut and open the dura and pia maters.
(e) Insert the array slowly and gently into the cortex.
(f) Close the dura over the array, leaving only a small incision where the ribbon cable exits.
(g) Close the skull window using dental acrylic.
(h) Secure the head stage amplifier with bone screws to the skull. Use dental acrylic to reinforce the bone screws.
(i) Stretch the flap of the skin over the replaced skull and suture it to close the incision.
(j) Keep the animal analgesics and antibiotics and allow it to recover for two weeks.

In-Vivo behavioral tests are based on delayed nonmatch-to-sample tests, which are described elsewhere [30]. The behavioral test can be used to validate and verify the system: (a) Train the animal to select a lever from n (e.g. 5) after hearing a tone. The animal must press a lever after hearing a tone to receive a sugar pellet. Only the first lever pressed is considered. Afterward, all the lever withdraw and can no longer be pressed until the next trial. (b) Initially the selection is based on the sound played. (c) After the animal become familiar with (a) and (b), the lever to be pressed is transmitted as an electrical stimulus pattern over the array. A tone not used in b) is played. (d) Animal behavioral performance in (c) and (a) is compared, as well as the chance of randomly guessing the correct lever (e.g. 20%). (e) The neural activity after presentation of the stimulus pattern is recorded by the electrode array and transmitted to a computer for analysis. (f) After termination of the experiment, the brain is fixed for histological analysis to determine the extent of integration between the brain and grafted cells.

### Features and Advantages:

High specificity: The main aspects of the devices and methods of using the devices provided herein, and advantage over conventional existing devices, are those that provide the extremely high specificity between the electric contact and target neurons. This is achieved by providing a brain-facing surface of the device that functionally acts as a ground in combination with the grafted cells in the wells, so that electrical current can only flow as action potentials to or from the grafted cells. The grafted cells, in turn, will only synapse with one or the few cells that they can physically reach. This combination of grounded tissue-facing surface, grafted cells in the wells, and physical connection between the grafted cells and biological cells of the tissue provides the high specificity.

Power Efficiency: Furthermore, there is about one cell, or at most several cells (e.g., less than 10, less than 5, or less than 3) per contact to the target biological tissue. Such a few number of cells that synaptically connect at a particular location provides a number of functional benefits, including a signal from each cell can be transformed into a spike train with a threshold circuit. This results in a greatly reduced power consumption by a device, including a device that is a head stage. Conventional head stages historically have a serious problem with power consumption, particularly for implantable/integrated head stages. The instant devices and methods have reduced power consumption, particularly as the devices and methods are effectively working with action potentials instead of analog systems with attendant transmission requirements.

Noise Resistance: because there is only one or a few cells in every well, every channel will pick up spike trains corresponding to these cells, which are in effect carrying only timing information. Noise from local field potentials that penetrates the wells cannot disrupt the operation because they distort the spike trains vertically rather than horizontally.

The cellular electrode can tolerate a large amount of noise before breaking down, making it tolerant to internal or external noises.

High Bandwidth: The first part of the fabrication process is the same for a Utah array. Therefore the instant devices can achieve the same array density as the number of needles in state-of-art Utah array. With a spacing of 100 μm, a 10×10 mm array can contain up to 10,000 unique contacts. Because electrical interconnects will be routed through multiple conductive polymer layers, this density is possible. In practice, however, the density may be limited to the channel count of the best amplifier systems.

The devices provided herein also have good temporal resolution. The response of grafted neurons serving as electrical connection can be predicted using the pulse waveform, cell type and well geometry.

The combination of high specificity and resolution allow high bandwidth communication, and facilitates transmission of meaningful encoded signal in time and space (like spike trains) between electronic devices and neural tissue.

Mechanical and Chemical Bio-Compatibility: The device is formed from the polymer, including from PDMS. With PDMS as a dominant component of the system, the device has excellent mechanical and chemical characteristics for biomedical applications, including interfacing with soft biological tissue. PDMS is an elastic material which can have a mechanical parameter, such as a Young's modulus, matched to the to-be-interfaced tissue. Therefore, there will be minimal abrasion between the electrode array and the neural tissue due to mismatch in mechanical properties. PDMS is also biologically inert, non-toxic and tends not to trigger immune responses. Accordingly, the device is suitable for long term use with biological tissue, including in vitro, ex vivo and in vivo.

Being a soft and flexible device, the device can also be rolled into a cuff electrode configuration, suitable for electrically interfacing with the spinal cord or peripheral nerves.

Histological Compatibility: PDMS is optically clear and so the device can be viewed directly with a transmission microscope. The device is sufficiently soft that it can be cut with a vibrotome and scanned by a confocal microscope. This makes the device an excellent solution for long-term in vitro studies, such as organotypic cultures.

Modular Compatibility: The device may be designed to incorporate head stage electronics. Alternatively, the device can also be made compatible with any amplifier that accepts a micro-ribbon connector. This can be done by bonding or directly patterning Parylene ribbons on the device.

To facilitate device characterization, the device may be made to have only 32 or 64 channels. This allows the device to be tested using existing TBSI or Multichannel system head stage amplifiers.

Low cost: Mass produced arrays are made with reusable masters and photo-reactive PDMS. This reduces cost at scale and makes the devices attractive for studies with small animals.

Computational Model for Design Iteration: The number of cells in each well is a Poisson distribution with the probability equal to plated cell density divided by the array density. This can be used to limit number of cell per well to several each. Cells processes largely develop in a vertical orientation along the collagen scaffold against oxygen and nutrient concentrations.

Detailed computational analysis for the reaction of neural tissue to injected charges is not possible in general due to the complexity of the neural tissue structure. The well-based design insulates the analysis of electrode system from the complexity of biological structures and makes the problem tractable because of the confining of electrical connection via the well and grounded nature of the device surface. A theoretical understanding of the system further facilitates particulars of the design in an iterative and/or simulation, or by an AI.

Segmented numerical model (with NEURON) can be created for dendrite and axon from grafted cells (following gradients) and towards grafted cells (against gradients). Simulations are performed to predict spike trains triggered by time-varying potential gradient (for stimulation) or the extracellular potentials generated by moving action potentials (for recording). These models are used to guide design parameters. Cell morphology obtained from confocal microscopes are used to verify the models.

The distribution of number of cells in wells is expected to be a Poisson distribution. Therefore, to limit the number of wells with multiple cells, a large number of wells may be empty. This situation can be improved by cell capture and handling techniques provided in various micro-fluid devices. [31]. In this manner, the number of cells per well is controlled via microfluidics. For example, cell sorters (see, e.g., fluorescent activated cell sorters FACS) plus fluidic controllers, positioners and the like can be used to provide precise number of cells per well. A FACS can provide a single cell from a nozzle. The nozzle can be connected to a custom setup on a floating air table, where a micro manipulator moves a petri dish containing the array at a fixed speed. Therefore, a single cell can be placed at each well, as desired.

Because neural signal are conducted mostly passively in dendrite and actively in axons, it is better for stimulation if grafted cells send axons into the adjacent tissue. Conversely it is better for recording if adjacent cells send axons to synapse onto grafted cells.

The devices described herein comprise an electrode array for a neural interface which achieves high specificity using grafted cells in concert with special conductive and non-conductive polymer layers and regions. In this manner, the devices and related methods of using the electrode array devices, outperform all systems currently on the market, including for chronic applications.

In another embodiment, a standard surface electrode array 240 (see, e.g., FIG. 1B) is fabricated on the tissue-facing layer with standard techniques, to provide the desired specificity and ability to obtain a bulk aggregate electrical interface with tissue. This secondary electrode array can be positioned on the top-most biological-facing tissue instead of part of the ground. This facilitates simultaneous collection of more “bulk” or “overall” electrical signals. This embodiment is particularly useful for debugging or serving as a back-up. Of course, there is an attendant impact with respect to ground being imperfect; the imperfect ground can be accommodated by increasing device thickness.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure.

Every device, system, formulation, combination of components, or method described or exemplified herein can be used to practice the invention, unless otherwise stated.

Whenever a range is given in the specification, for example, a size range, a density range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

[1] J. Park, S. Kim, J. Li, and A. Han, “Axon length quantification microfluidic culture platform for growth and regeneration study,” Methods in Molecular Biology, vol. 1162. pp. 85-95, 2014. doi: 10.1007/978-1-4939-0777-9 7.
[2] E. Malishev et al., “Microfluidic device for unidirectional axon growth,” J. Phys. Conf. Ser., vol. 643, no. 1, p. 012025, October 2015, doi: 10.1088/1742-6596/643/1/012025.
[3] A. S. Asan, S. G. Id, and M. Sahin, “Electrical fields induced inside the rat brain with skin, skull, and dural placements of the current injection electrode,” pp. 1-15, 2019.
[4] A. M. Turner and W. T. Greenough, “Differential rearing effects on rat visual cortex synapses. I. Synaptic and neuronal density and synapses per neuron,” Brain Res., vol. 329, no. 1-2, pp. 195-203, 1985, doi: 10.1016/0006-8993 (85) 90525-6.
[5] M. Chernov and A. W. Roe, “Infrared neural stimulation: a new stimulation tool for central nervous system applications,” Neurophotonics, vol. 1, no. 1, p. 011011, 2014, doi: 10.1117/1.nph.1.1.011011.
[6] C. Seok, F. Y. Yamaner, M. Sahin, and O. Oralkan, “A Sub-Millimeter Lateral Resolution Ultrasonic Beamforming System for Brain Stimulation in Behaving Animals,” Proc. Annu. Int. Conf. IEEE Eng. Med. Biol. Soc. EMBS, pp. 6462-6465 July 2019, doi: 10.1109/EMBC.2019.8857627.
[7] H. Nollet, L. Van Ham, P. Deprez, and G. Vanderstraeten, “Transcranial magnetic stimulation: review of the technique, basic principles and applications,” Vet. J., vol. 166, no. 1, pp. 28-42, July 2003, doi: 10.1016/S1090-0233 (03) 00025-X.
[8] L. Huber et al., “Ultra-high resolution blood volume fMRI and BOLD fMRI in humans at 9.4 T: Capabilities and Challenges,” Neuroimage, vol. 178, p. 769, September 2018, doi: 10.1016/J.NEUROIMAGE.2018.06.025.
[9] J. H. Duyn, “The future of ultra-high field MRI and fMRI for study of the human brain,” Neuroimage, vol. 62, no. 2, p. 1241 August 2012, doi: 10.1016/J.NEUROIMAGE.2011.10.065.
[10] J. Wu et al., “Kilohertz two-photon fluorescence microscopy imaging of neural activity in vivo,” Nat. Methods, vol. 17, no. 3, p. 287, March 2020, doi: 10.1038/S41592-020-0762-7.
[11] S. M. Rajguru, A. I. Matic, and C. P. Richter, “Optical Stimulation of Neurons,” Laser Imaging Manip. Cell Biol. pp. 99-112, 2010, doi: 10.1002/9783527632053.ch6.
[12] E. Musk, “An integrated brain-machine interface platform with thousands of channels”. [13] K. E. Jones, P. K. Campbell, and R. A. Normann, “A Glass/Silicon Composite Intracortical Electrode Array,” pp. 423-437, 1992.
[14] R. Bhandari, S. Negi, and F. Solzbacher, “Wafer-scale fabrication of penetrating neural microelectrode arrays,” pp. 797-807, 2010, doi: 10.1007/s10544-010-9434-1.
[15] R. Luo, H. Li, B. Du, S. Zhou, and Y. Zhu, “A simple strategy for high stretchable, flexible and conductive polymer films based on PEDOT: PSS-PDMS blends,” Org. Electron., vol. 76, no. April 2019, p.105451, 2020, doi: 10.1016/j.orgel.2019.105451.
[16] R. K. Pal, S. Pradhan, L. Narayanan, and V. K. Yadavalli, “Micropatterned conductive polymer biosensors on flexible PDMS film,” vol. 259, pp. 498-504, April 2018, Accessed: Mar. 15, 2022. [Online]. Available: http://dx.doi.org/10.1016/j.snb.2017.12.082
[17] B. H. Cong and T. Pan, “Photopatternable Conductive PDMS Materials for Microfabrication,” pp. 1912-1921, 2008, doi: 10.1002/adfm.200701437.
[18] B. X. Niu, S. Peng, L. Liu, W. Wen, and P. Sheng, “Characterizing and Patterning of PDMS-Based Conducting Composites **,” pp. 2682-2686, 2007, doi: 10.1002/adma.200602515.
[19] C. Lang and D. Boning, “Spin Coating Modeling and Planarization Using Fill Patterns for Advanced Packaging Technologies,” pp. 192-197, 2017.
[20] L. M. Peurrung and D. B. Graves, “Spin Coating Over Topography,” vol. 6, no. 9205040, 1993.
[21] J. Garra, G. Advanced, and J. Currie, “Dry etching of polydimethylsiloxane for microfluidic systems,” pp. 975-982, 2002, doi: 10.1116/1.1460896.
[22] M. Zhang and P. Watson, “ScholarlyCommonsReactive Ion Etching Selectivity of Si/SiO2: Comparing of two fluorocarbon gases CHF3 and CF4 Reactive Ion Etching Selectivity of Si/SiO2: Comparing of two fluorocarbon gases,” pp. 4-9, 2019.
[23] S. A. Gupta and R. K. Gupta, “A Parametric Study of Spin Coating over Topography+,” vol. 5885, no. 97, pp. 2223-2227, 1998.
[24] E. Smela, “Patterning PDMS using a combination of wet and dry etching Patterning PDMS using a combination of wet and dry etching”, doi: 10.1088/0960-317/19/4/047002.
[25] N. R. A. M. Ejia, “PLURIPOTENT STEM CELLS Concise Review: Generation of Neurons From Somatic Cells of Healthy Individuals and Neurological Patients Through Induced Pluripotency or Direct Conversion,” pp. 2811-2817, 2014.
[26] H. Kanno, “Regenerative therapy for neuronal diseases with transplantation of somatic stem cells,” vol. 5, no. 4, pp. 163-171, 2013, doi: 10.4252/wjsc.v5.14.163.
[27] D. Bodas and C. Khan-malek, “Formation of more stable hydrophilic surfaces of PDMS by plasma and chemical treatments,” vol. 83, pp. 1277-1279, 2006, doi: 10.1016/j.mee.2006.01.195.
[28] E. H. Chang, S. A. Frattini, S. Robbiati, and P. T. Huerta, “Construction of Microdrive Arrays for Chronic Neural Recordings in Awake Behaving Mice,” JoVE (Journal Vis. Exp., no. 77, p. e50470, July 2013, doi: 10.3791/50470.
[29] “The cerebral cortex of the rat.—PsycNET.” https://psycnet.apa.org/record/1990-98262-000 (accessed May 20, 2022).
[30] S. A. Deadwyler, T. Bunn, and R. E. Hampson, “Hippocampal ensemble activity during spatial delayed-nonmatch-to-sample performance in rats,” J. Neurosci., vol. 16, no. 1, pp. 354-372, January 1996, doi: 10.1523/JNEUROSCI.16-01-00354.1996.
[31] P. Kim, K. W. Kwon, M. C. Park, S. H. Lee, and S. M. Kim, “Soft Lithography for Microfluidics: a Review,” vol. 2, no. 1, pp. 1-11, 2008.
[32] X. Cheng, C. Bao, X. Wang, F. Zhang, and W. Dong, “Soft surface electrode based on PDMS-CB conductive polymer for electrocardiogram recordings,” Appl. Phys. A, pp. 1-7, 2019, doi: 10.1007/s00339-019-3124-5.
[33] C. Liu, “Microfabrication of conductive polymer nanocomposite for sensor applications,” 2012.
[34] R. Karimi-Chaleshtori, A. H. Nassajpour-Esfahani, M. R. Saeri, P. Rezai, and A. Doostmohammadi, “Silver nanowire-embedded PDMS with high electrical conductivity: nanowires synthesis, composite processing and electrical analysis,” Mater. Today Chem., vol. 21, p. 100496, August 2021, doi: 10.1016/J.MTCHEM.2021.100496.
[35] W. Chen, R. H. W. Lam, and J. Fu, “Photolithographic surface micromachining of polydimethylsiloxane (PDMS),” Lab Chip, vol. 12, no. 2, pp. 391-395, December 2011, doi: 10.1039/C1LC20721K.
[36] M. Redecker et al., “Topographical Micropatterning of Poly (dimethylsiloxane) Using Laminar Flows of Liquids in Capillaries **,” Electrochem. Methods & Fundamentals Appl., vol. 76, 2000.
[37] J. H. Kim et al., “Simple and cost-effective method of highly conductive and elastic carbon nanotube/polydimethylsiloxane composite for wearable electronics,” Sci. Reports 2018 81, vol. 8, no. 1, pp. 1-11, January 2018, doi: 10.1038/s41598-017-18209-w.
[38] S. Z. Szilasi and L. Juhasz, “Selective etching of PDMS: etching as a negative tone resist,” pp. 1-16.
[39] B. Gale, B. Gale, M. A. Eddings, M. A. Johnson, and B. K. Gale, “Determining the optimal PDMS-PDMS bonding technique for microfluidic devices Determining the optimal PDMS-PDMS bonding technique for microfluidic devices”, doi: 10.1088/0960-1317/18/6/067001.
[40] C. Chen and K. Wharton, “RSC Advances Characterization and failure mode analyses of air plasma oxidized PDMS-PDMS bonding by peel testing+,” pp. 1286-1289, 2017, doi: 10.1039/c6ra25947b.
[41] D. C. Duffy, J. C. Mcdonald, O. J. A. Schueller, and G. M. Whitesides, “Rapid Prototyping of Microfluidic Systems in Poly (dimethylsiloxane),” vol. 70, no. 23, pp. 4974-4984, 1998.
[42] P. R. Ray et al., “RNA profiling of human dorsal root ganglia reveals sex differences in mechanisms promoting neuropathic pain,” Brain, vol. 146, no. 2, pp. 749-766, February 2023, doi: 10.1093/BRAIN/AWAC266.
[43] A. Björklund et al., “Neural transplantation for the treatment of Parkinson's disease,” Lancet Neurol., vol. 2, no. 7, pp. 437-445, July 2003, doi: 10.1016/S1474-4422 (03) 00442-3.
[44] D. G. Southwell, R. C. Froemke, A. Alvarez-Buylla, M. P. Stryker, and S. P. Gandhi, “Cortical Plasticity Induced by Inhibitory Neuron Transplantation,” Science, vol. 327, no. 5969, p. 1145 Feb. 2010, doi: 10.1126/SCIENCE.1183962.
[45] X. Yin, J. C. Xu, G. S. Cho, C. Kwon, T. M. Dawson, and V. L. Dawson, “Neurons Derived from Human Induced Pluripotent Stem Cells Integrate into Rat Brain Circuits and Maintain Both Excitatory and Inhibitory Synaptic Activities,” eNeuro, vol. 6, no. 4, July 2019, doi: 10.1523/ENEURO.0148-19.2019.
[46] K. Takahashi et al., “Induction of Pluripotent Stem Cells from Adult Human Fibroblasts by Defined Factors,” Cell, vol. 131, no. 5, pp. 861-872, November 2007, doi: 10.1016/j.cell.2007.11.019.
[47] N. Malik and M. S. Rao, “A Review of the Methods for Human iPSC Derivation”, doi: 10.1007/978-1-62703-348-O_3.
[48] M. Frega et al., “Rapid Neuronal Differentiation of Induced Pluripotent Stem Cells for Measuring Network Activity on Micro-electrode Arrays,” JoVE (Journal Vis. Exp., vol. 2017, no. 119, p. e54900, January 2017, doi: 10.3791/54900.
[49] J. R. Loverde, V. C. Ozoka, R. Aquino, L. Lin, and B. J. Pfister, “Live Imaging of Axon Stretch Growth in Embryonic and Adult Neurons,” https://home.liebertpub.com/neu, vol. 28, no. 11, pp. 2389-243 Nov. 2011, doi: 10.1089/NEU.2010.1598.
[50] G. C. Magou, B. J. Pfister, and J. R. Berlin, “Effect of acute stretch injury on action potential and network activity of rat neocortical neurons in culture,” Brain Res., vol. 1624, pp. 525-535, October 2015, doi: 10.1016/J.BRAINRES.2015.07.056.
[51] S. Bhattacharya, A. Datta, J. M. Berg, and S. Gangopadhyay, “Studies on surface wettability of poly(dimethyl) siloxane (PDMS) and glass under oxygen-plasma treatment and correlation with bond strength,” J. Microelectromechanical Syst., vol. 14, no. 3, pp. 590-597, June 2005, doi: 10.1109/JMEMS.2005.844746.
[52] S. Gok and M. Sahin, “Rat forelimb movement components segregated by corticospinal tract activity,” in International IEEE/EMBS Conference on Neural Engineering, NER, 2017, pp. 312-315. doi: 10.1109/NER.2017.8008353.
[53] C.R. Vistas et al. “Silanization of glass chips-A factorial approach for optimization.” Applied Surface Science 286:314-315 (December 2013). C. Moraes et al. “Solving the shrinkage-induced PDMS alignment registration issue in multilayer soft lithography.” J. Micromech. Microeng. 19 (2009) (6 pp.).

1. An electrode comprising:
an electrically conducting material having an outer surface, wherein the outer surface is optionally formed of an outer material that is different from a core material of the electrically conducting material;
at least one neuronal cell grafted to the outer surface of the electrically conducting material; and
a biological electrical connection between the grafted neuronal cell and a target neuronal cell in a biological tissue in which the electrode is positioned, wherein substantially all electrical connection between the electrode and the biological tissue is through the biological electrical connection.

2. A cellular electrode array comprising:
a plurality of electrodes of claim 1;
a flexible polymer substrate having a top surface;
an array of wells disposed in the flexible polymer substrate, each well having:
an inner-facing surface that defines a well volume, the inner facing surface comprising a bottom surface and side wall surfaces that extend from the bottom surface to the flexible polymer substrate top surface;
the electrically conducting material forming a conductive contact, optionally a polymer conductive contact, at least partially covering the well inner-facing surface;
a well depth corresponding to a distance between the flexible polymer substrate top surface and the well bottom surface;
a hydrogel disposed in each well;
wherein each well is configured to receive a neuronal cell to form the grafted neuronal cell to electrically connect to a neuronal cell in the biological tissue, and the hydrogel is configured to provide directed growth of sprouted axons and dendrites from the neuronal cell in a direction toward the flexible polymer substrate top surface.

3. The cellular electrode array of claim 2, further comprising at least one neuronal cell in each of the wells, including an average of between 1 and 5 neuronal cells per well.

4. The cellular electrode array of claim 2, wherein the conductive polymer contact is positioned at the well bottom surface.

5. The cellular electrode array of claim 2, configured to operably connect flush against a tissue surface, including a tissue that corresponds to a brain surface with the operably connected mediated by synaptic connections between the neuronal cells in the wells and dendrites in the brain adjacent to the brain surface.

6. The cellular electrode array of claim 2, wherein:
each well has a well depth that is between 10 μm and 2 mm;
each well has a volume that is between 10 μL and 1 mL;
each well has a characteristic diameter of between 1 μm and 1 mm;
a well density of between 0.001 wells/μm² (1000 wells/mm²) to 0.1 wells/μm² (10⁵ wells/mm²); and/or
adjacent wells are separated by a separation distance of between 10 μm and 1 mm.

7. (canceled)

8. (canceled)

9. (canceled)

10. (canceled)

11. The cellular electrode array of claim 2, wherein the polymer substrate is conformable to a tissue surface.

12. The cellular electrode array of claim 2, wherein the conductive polymer contact has a thickness of between 1 μm and 100 μm and/or greater than the well depth.

13. The cellular electrode array of claim 2, wherein the flexible polymer substrate comprises a plurality of polydimethylsiloxane (PDMS) layers, including a conductive PDMS layer and a non-conductive PDMS layer; wherein the non-conductive PDMS layer on the conductive PDMS layer forms an insulated conductive layer that is an electrical interconnect between the electrodes and external electronic components; wherein the conductive PDMS layer is a PDMS composite layer.

14. (canceled)

15. The cellular electrode array of claim 2 comprising at least two non-conductive PDMS layers.

16. The cellular electrode array of claim 2, further comprising the neuronal cells grafted onto the conductive polymer contact; wherein:
the neuronal cells are selected to synaptically connect to a neural tissue during use; and
are immune compatible with a subject having the neural tissue for in vivo use or with the subject from which neural tissue is obtained for in vitro research use.

17. (canceled)

18. (canceled)

19. The cellular electrode array of claim 2, wherein the top surface of the flexible polymer is an electrical conductor to ground the cellular electrode array so that during use electrical communication between the cellular electrode array and a subject is confined to the neuronal cells in the wells synaptically connected to the subject (in vivo) or to a volume of an explanted tissue (in vitro or ex vivo); and having a device footprint of up to 1000 mm², and optionally with up to 10,000 unique contacts between the neuronal cells in the wells and a tissue in which the cellular electrode array is implanted.

20. (canceled)

21. The cellular electrode array of claim 2, wherein the top surface comprises a surface electrode array for bulk electrical interfacing with a biological tissue.

22. A method of electrically interfacing with biological tissue, the method comprising the steps of:
providing the electrode or the cellular electrode array of claim 1;
grafting a neuronal cell to the electrode or in each of the wells by providing less than 10 neuronal cells to the electrode or to each of the wells and culturing the neuronal cells to form axons and/or dendrites;
contacting the grafted neuronal cells to the biological tissue;
electrically connecting at least a portion of the grafted neuronal cells to biological cells adjacent to the grafted neuronal cells, thereby forming electrical contacts between the biological tissue and the cellular electrode array; and thereby electrically interfacing with the biological tissue via the grafted neuronal cells that are in electrical contact with the biological tissue.

23. The method of claim 22, wherein the electrical interfacing comprises electrically actuating the biological tissue and/or measuring an electrical parameter of the biological tissue.

24. The method of claim 22, wherein the electrically interfacing step comprises measuring current flowing to or from at least a portion of the grafted neuronal cells that are electrically connected to biological tissue; and further comprising the steps of:
selecting a portion of the wells having neuronal cells electrically connected to biological cells; and
electrically interfacing the selected portion of the wells with the biological cells;
wherein the selected portion of wells corresponds to a spatial region having a surface area of between 1 μm² and 5 mm².

25. (canceled)

26. (canceled)

27. The method of claim 22, further comprising the step of matching a mechanical property of the cellular electrode array with a mechanical property of the biological tissue; wherein the biological tissue is selected from the group consisting of: brain, spinal cord, peripheral nerves, a three-dimensional cell culture (including a bioartificial organ), and organotypic cultures.

28. (canceled)

29. The method of claim 22, wherein at least 80% of a total electric current between the cellular electrode array and surrounding electrically connected tissue is via the grafted neuronal cells.

30. A method of making a grafted cellular electrode array, the method comprising the steps of:
providing the cellular electrode array of claim 1;
introducing at least one neuronal cell to at least one well;
culturing the at least one neuronal cell for a time period selected to ensure:
attachment the at least one neuronal cell to a bottom surface and/or a sidewall of the well, or the hydrogel scaffold;
sprouting axons and/or dendrites from the at least one neuronal cell,
thereby grafting the neuronal cell to the well and making the grafted cellular electrode array.
